(19) United States
(12) Patent Application Publication
(10) Pub. No.: US 2025/0152795 A1
(43) Pub. Date: May 15, 2025

(54) APPARATUS FOR EXTRACORPOREAL BLOOD TREATMENT

(71) Applicant: GAMBRO LUNDIA AB, Lund (SE)

(72) Inventor: Dominique Pouchoulin, Tramoyes (FR)

(21) Appl. No.: 18/722,260

(22) PCT Filed: Dec. 22, 2022
(86) PCT No.: PCT/EP2022/087538

§ 371 Date: Jun. 20, 2024

(30) Foreign Application Priority Data
Dec. 30, 2021 (EP) 21306959.4

(51) Int. Cl. A61M 1/36; G16H 40/63

(52) U.S. Cl. CPC A61M 1/3656; G16H 40/63; A61M 2202/0021; A61M 2202/0413; A61M 2205/10; A61M 2205/18; A61M 2205/3303; A61M 2205/3327; A61M 2205/3334; A61M 2205/50; A61M 2205/75; A61M 2230/30

(57) ABSTRACT

An apparatus for extracorporeal blood treatment is configured for detecting a disconnection between a connector (6a, 7a) for connection to a vascular access device (400) fixed to a patient (P) and the vascular access device (400) by: calculating a hydrostatic pressure difference (P_{H_patient}) due to a difference in height between the vascular access device (400) and a pressure sensor (25, 26); calculating a section pressure drop (ΔPₗᵢₙₑ) due to a section of the blood circuit from the connector (6a, 7a) to the pressure sensor (25, 26); calculating a disconnection pressure (P_{disc}) from the hydrostatic pressure difference (P_{H_patient}) and the section pressure drop (ΔPₗᵢₙₑ); receiving a measured pressure (P) from the pressure sensor (25, 26); detecting a disconnection of the connector (6a, 7a) from the vascular access device (400) by comparing the measured pressure (P) with a pressure alarm threshold (Pₜₕᵣₑₛₕ) function of the disconnection pressure (P_{disc}).

### TECHNICAL FIELD

The present invention relates to an apparatus for extracorporeal blood treatment capable of detecting disconnection events during treatment. For instance, the present invention is applicable in the context of medical apparatuses and methods for continuous renal replacement therapies (CRRT) or other therapies in Intensive Care Units (ICU), like Extra-Corporeal CO2 Removal (ECCO2R), Hemo-Perfusion (HP) or Therapeutic Plasma Exchange (TPE).

### BACKGROUND OF THE INVENTION

CRRT systems are configured for delivering treatments designed for patients versing in acute states of illness and who have temporarily lost their kidney function in its entirety. CRRT monitors should be able to deliver various therapies, e.g.: ultrafiltration (UF), continuous veno venous hemofiltration (CCVH), continuous veno venous hemodiafiltration (CVVHDF), continuous veno venous hemodialysis (CCVHD).

Within the context of CRRT, in which the vascular access is typically performed via a catheter, the disconnection of the line or lines from the catheter may occur and is critical for the risk of blood loss or air embolism. Indeed, return line disconnection events with no detection from the system may lead to patient death by exsanguination driven by the blood pump. Known systems for the detection of return line disconnection are based on monitoring the return pressure and on the assessment of at least one of the following conditions:
decrease of the return pressure from a reference value (operating point) by more than X mmHg, wherein the value of X can be automatically set by the system and/or be adjusted by an operator;
return pressure below a fixed threshold value Y, which could be again automatically set by the system or be operator adjustable.

Experience indicates that above design is not reliable, since most alarm occurrences do not match with disconnection events and some actual disconnection events are not detected.

Document WO2018053461A1 is also known, which discloses a method and a system for detecting a condition indicative of a dislodged needle in a hemodialysis procedure. A venous return pressure for a patient undergoing dialysis is measured. The venous return pressure is analyzed via a controller and an intravascular blood pressure in proximity to a location of needle insertion into the patient is derived. A lower limit is calculated as a function of the intravascular blood pressure via the controller. An average of the venous return pressure is calculated via the controller during a predetermined time window. The average is compared to the lower limit via the controller and, if the average is within a range of the lower limit, the controller determines that a condition indicative of a dislodged needle is present.

Document US20160354531A1 discloses a method and a device for monitoring a vascular access during an extracorporeal blood treatment. The method and the device are based on the monitoring of the difference between the venous pressure measured by a venous pressure sensor and the arterial pressure measured by an arterial pressure sensor in the extracorporeal blood circuit. A test function describing disturbances in the extracorporeal blood circuit is determined. The test function is used to determine a noise-free differential pressure from the measured venous and arterial pressure, the differential pressure being evaluated in an arithmetic and evaluation unit to identify a defective vascular access.

Document US20180126062A1 discloses a monitoring system performing a method for detecting a disruption of a fluid connection between a first fluid containing system and a second fluid containing system. The monitoring system may be connected to or may be part of an apparatus for blood treatment and operable to detect a disconnection of an extracorporeal blood circuit from a vascular system of a patient. The monitoring system generates a monitoring signal, which is representative of a fluid pressure in respect of the first fluid containing system and which is responsive to the disruption of the fluid connection, and a tracking signal which corresponds to and is more smoothed over time than the monitoring signal. The monitoring system further sets a detection range in a given relation to the tracking signal so that the detection range follows changes in the tracking signal and detects a condition indicative of the disruption by comparing a current pressure value of the monitoring signal to the detection range.

### GLOSSARY

The following terms/parameters are consistently used throughout the equations provided in the following description and in the appended claims.

Terms/Parameters

| | |
|---|---|
| P_{H}_{_{—}}ₚₐₜᵢₑₙₜ | hydrostatic pressure difference |
| P_{Hret}_{_{—}}ₚₐₜᵢₑₙₜ | hydrostatic pressure difference return line |
| P_{Hwith}_{_{—}}ₚₐₜᵢₑₙₜ | hydrostatic pressure difference withdrawal line |
| ΔPₗᵢₙₑ | section pressure drop |
| ΔPᵣₑₜ_{_{—}}ₗᵢₙₑ | section pressure drop return line |
| ΔP_{with}_{_{—}}ₗᵢₙₑ | section pressure drop withdrawal line |
| P_{disc} | disconnection pressure |
| Pᵣₑₜ_{_{—}}_{disc} | return disconnection pressure |
| P_{with}_{_{—}}_{disc} | withdrawal disconnection pressure |
| Pₜₕᵣₑₛₕ | pressure alarm threshold |
| Pᵣₑₜ_{_{—}}ₜₕᵣₑₛₕ | pressure alarm threshold return line |
| P_{with}_{_{—}}ₜₕᵣₑₛₕ | pressure alarm threshold withdrawal line |
| P | measured pressure |
| Pᵣₑₜ | measured pressure return line |
| P_{with} | measured pressure withdrawal line |
| P_{offset} | offset pressure |
| P_{offset}_{_{—}}ᵣₑₜ | offset pressure return line |
| P_{offset}_{_{—}}_{with} | offset pressure withdrawal line |
| P_{Qb0} | static pressure |
| Pᵣₑₜ_{_{—}}_{Qb0} | static pressure return line |
| P_{with}_{_{—}}_{Qb0} | static pressure withdrawal line |
| ΔP_{cath} | pressure drop catheter |
| ΔPᵣₑₜ_{_{—}}_{cath} | pressure drop catheter return line |
| ΔP_{with}_{_{—}}_{cath} | pressure drop catheter withdrawal line |
| ΔP_{safety} | safety margin |
| ΔPᵣₑₜ_{_{—}}_{safety} | safety margin return line |
| ΔP_{with}_{_{—}}_{safety} | safety margin withdrawal line |
| Pᵥₑₙₒᵤₛ | patient central venous pressure |
| Qb | blood flow rate |
| Qb0 | zero blood flow rate |
| H | difference in height |
| Hᵣₑₜ | difference in height return pressure sensor |
| H_{with} | difference in height withdrawal pressure sensor |
| kₗᵢₙₑ | section pressure drop coefficient |
| kᵣₑₜ_{_{—}}ₗᵢₙₑ | section pressure drop coefficient return line |
| k_{with}_{_{—}}ₗᵢₙₑ | section pressure drop coefficient withdrawal line |
| L | length of the section |
| d | internal diameter of the section |
| L_{with} | length of the section return line |
| d_{with} | internal diameter of the section return line |
| Lᵣₑₜ | length of the section withdrawal line |
| dᵣₑₜ | internal diameter of the section withdrawal line |
| μ | blood viscosity |

### SUMMARY

In this situation, it is a general object of the present invention to offer a technical solution capable overcoming one or more of the above drawbacks.

It is an object of the present invention to improve the reliability of detection of events of disconnection of the blood circuit (i.e. return line and/or withdrawal line) from the patient during extracorporeal blood treatments and thus improving safety of the patient.

More in detail, it is an aim of the present invention to provide an apparatus and a method allowing to detect the actual disconnection events and to reduce occurrence of false alarms. It is also an aim of the present invention to manage clotting events which may develop along the therapy.

It is also an aim of the present invention to provide a reliable detection system which does not have a negative impact on costs of the apparatus and/or of the treatments.

It is also an aim of the present invention to provide a reliable detection system which may be easily implemented in current extracorporeal blood treatment apparatuses and which does not require improvements of the hardware of these apparatuses.

At least one of the above objects is substantially reached by an apparatus for extracorporeal blood treatment according to one or more of the appended claims.

One or more of the above objects is also substantially reached by a method of detecting disconnection events in an apparatus for extracorporeal blood treatment.

Apparatus and method according to aspects of the invention are here below described.

A 1^{st} independent aspect concerns an apparatus for extracorporeal blood treatment, comprising:

a filtration unit;

an extracorporeal blood circuit having a blood withdrawal line connected to an inlet of the filtration unit and a blood return line connected to an outlet of the filtration unit, said extracorporeal blood circuit being configured for connection to a cardiovascular system of a patient; the extracorporeal blood circuit comprising at least one connector for connection to a vascular access device fixed to the patient;

at least one pressure sensor configured to detect a pressure in at least one measurement location in the extracorporeal blood circuit;

a blood pump configured to control the flow of blood through the extracorporeal blood circuit;

a control unit connected to the blood pump and to the at least one pressure sensor, the control unit being configured for detecting a disconnection between the at least one connector of the extracorporeal blood circuit and the vascular access device, by:
calculating a hydrostatic pressure difference P_{H_patient} due to a difference in height H between the vascular access device and said at least one pressure sensor;
calculating a section pressure drop ΔPₗᵢₙₑ due to a section of the blood circuit from the connector to said at least one pressure sensor;
calculating a disconnection pressure P_{disc} as an algebraic sum of the hydrostatic pressure difference P_{H_patient} and the section pressure drop ΔPₗᵢₙₑ;
setting a pressure alarm threshold Pₜₕᵣₑₛₕ as a function of the disconnection pressure P_{disc};
   wherein, during an extracorporeal blood treatment, the control unit is configured for:
receiving a measured pressure P from the at least one pressure sensor;
detecting a disconnection of the least one connector from the vascular access device by comparing the measured pressure P with the pressure alarm threshold Pₜₕᵣₑₛₕ.

A 1^{st} bis independent aspect is directed to a method of detecting disconnection events in an apparatus for extracorporeal blood treatment, wherein the apparatus comprises:

a filtration unit;

an extracorporeal blood circuit having a blood withdrawal line connected to an inlet of the filtration unit and a blood return line connected to an outlet of the filtration unit, said extracorporeal blood circuit being configured for connection to a patient cardiovascular system; the extracorporeal blood circuit comprising at least one connector for connection to a vascular access device fixed to the patient;

at least one pressure sensor configured to detect a pressure in at least one measurement location in the extracorporeal blood circuit;

a blood pump configured to control the flow of blood through the extracorporeal blood circuit;

wherein the method comprises:
calculating a hydrostatic pressure difference P_{H_patient} due to a difference in height H between the vascular access device and said at least one pressure sensor;
calculating a section pressure drop ΔPₗᵢₙₑ due to a section of the blood circuit from the connector to said at least one pressure sensor;
calculating a disconnection pressure P_{disc} as an algebraic sum of the hydrostatic pressure difference P_{H_patient} and the section pressure drop ΔPₗᵢₙₑ;
setting a pressure alarm threshold Pₜₕᵣₑₛₕ as a function of the disconnection pressure P_{disc};
receiving a measured pressure P from the at least one pressure sensor;
detecting a disconnection of the least one connector from the vascular access device by comparing the pressure P measured during the extracorporeal blood treatment with the pressure alarm threshold Pₜₕᵣₑₛₕ.

In a 2^{nd} aspect according to any one of the preceding two independent aspects, the disconnection pressure P_{disc} is a constant along at least part of the extracorporeal blood treatment.

In a 3^{rd} aspect according to any one of the preceding two independent aspects, the disconnection pressure P_{disc} is continuously updated along at least part of the extracorporeal blood treatment.

In a 4^{th} aspect according to any one of the preceding aspects, the calculation of the hydrostatic pressure difference P_{H_patient} is performed before starting the extracorporeal blood treatment.

In a 5^{th} aspect according to any one of the preceding aspects, the calculation of the hydrostatic pressure difference P_{H_patient} is repeated at least one time along the extracorporeal blood treatment, optionally every 6 to 12 hours, optionally after any change in bed height and/or patient position.

In a 6^{th} aspect according to any one of the preceding aspects, the calculation of the section pressure drop ΔPₗᵢₙₑ is performed before starting the extracorporeal blood treatment.

In a 7^{th} aspect according to any one of the preceding aspects, the calculation of the section pressure drop ΔPₗᵢₙₑ is repeated at least one time along the extracorporeal blood treatment.

In a 8^{th} aspect according to any one of the preceding seven aspects, the section pressure drop ΔPₗᵢₙₑ is continuously updated along at least part of the extracorporeal blood treatment.

In a 9^{th} aspect according to any one of the preceding aspects, in order to calculate the hydrostatic pressure difference P_{H_patient}, the control unit is configured for or the method comprises:
stopping the blood pump or keeping the blood pump stopped to obtain a zero a blood flow rate Qb0;
receiving or measuring a static pressure P_{Qb0} from the at least one pressure sensor while the blood flow rate is zero Qb0;
receiving or measuring a patient central venous pressure Pᵥₑₙₒᵤₛ;
calculating the hydrostatic pressure difference P_{H_patient} as difference between the static return pressure P_{Qb0} and the central venous pressure Pᵥₑₙₒᵤₛ;
   optionally the static pressure P_{Qb0} is received or measured when becoming stable after stopping the blood pump or keeping the blood pump stopped.

In a 10^{th} aspect according to the preceding aspects 9, the patient central venous pressure Pᵥₑₙₒᵤₛ is measured.

In an 11^{th} aspect according to the preceding aspect 10, the patient central venous pressure Pᵥₑₙₒᵤₛ is set as a default value, optionally between +6 mmHg and +12 mmHg (+800 Pa and +1600 Pa), optionally of +10 mmHg (+1333 Pa).

In a 12^{th} aspect according to any one of the preceding aspects, in order to calculate the section pressure drop ΔPₗᵢₙₑ, the control unit is configured for or the method comprises:
receiving a blood flow rate Qb;
receiving or calculating a blood viscosity μ;
receiving or calculating a section pressure drop coefficient kₗᵢₙₑ function of a geometry of the section of the blood circuit;
calculating the section pressure drop ΔPₗᵢₙₑ as function of the blood flow rate Qb, the blood viscosity μ and said section pressure drop coefficient kₗᵢₙₑ.

In a 13^{th} aspect according to the preceding aspect 12, the section pressure drop coefficient kₗᵢₙₑ is a constant.

In a 14^{th} aspect according to the preceding aspect 12 or 13, the section pressure drop coefficient kₗᵢₙₑ is given by the following equation: kₗᵢₙₑ=(128×L)/(π×d⁴); where L is a length of the section and d is an internal diameter of the section.

In a 15^{th} aspect according to the preceding aspect 12, the control unit is configured for or the method comprises: updating the section pressure drop coefficient kₗᵢₙₑ and consequently also the disconnection pressure P_{disc} during the extracorporeal blood treatment; optionally the section pressure drop coefficient kₗᵢₙₑ is continuously updated during the extracorporeal blood treatment, optionally every 1 to 10 minutes.

In a 16^{th} aspect according to the preceding aspect 15, an initial section pressure drop coefficient kₗᵢₙₑ^{init} at the start of the extracorporeal blood treatment is given, optionally by the following equation: kₗᵢₙₑ^{init}=(128×L)/(π×d⁴), and it is set as design section pressure drop coefficient kₗᵢₙₑ^{design}; where L is a length of the section and d is an internal diameter of the section.

In a 16^{th} bis aspect according to the preceding aspect 16, the initial section pressure drop coefficient kₗᵢₙₑ^{init} at the start of the extracorporeal blood treatment is derived from experimental measurements.

In a 17^{th} aspect according to the preceding aspect 16 or 16 bis, in order to update the section pressure drop coefficient kₗᵢₙₑ during the extracorporeal blood treatment, the control unit is configured for or the method comprises:
calculating an initial circuit pressure drop coefficient k_{circ}^{init} as k_{circ}^{init}=|(P^{init}−P_{Qb0})|/(μ×Qb) and set it as reference circuit pressure drop coefficient k_{circ}^{ref};
calculating an initial catheter pressure drop coefficient k_{cath}^{init} as k_{cath}^{init}=k_{circ}^{init}−kₗᵢₙₑ^{design} and set it as reference catheter pressure drop coefficient k_{cath}^{ref};
during the extracorporeal blood treatment, measuring the pressure P and calculating the circuit pressure drop coefficient as k_{circ}=(P−P_{Qb0})/(μ×Qb);
comparing each new value of the circuit pressure drop coefficient k_{circ} to the reference circuit pressure drop coefficient k_{circ}^{ref}.

In a 17^{th} bis aspect, the circuit pressure drop coefficient k_{circ} is the coefficient linked to the pressure drop due to the vascular access device and to the section of the blood circuit from the connector to said at least one pressure sensor.

In a 18^{th} aspect according to the preceding aspect 17, if k_{circ} is greater than k_{circ}^{ref}, then the section pressure drop coefficient kₗᵢₙₑ is updated as kₗᵢₙₑ=k_{circ}−k_{cath}^{ref} and the disconnection pressure P_{disc} is updated accordingly.

In a 19^{th} aspect according to the preceding aspect 17, if k_{circ} is less than k_{circ}^{ref}, then the section pressure drop coefficient kₗᵢₙₑ remains unchanged and the disconnection pressure P_{disc} remains unchanged.

In a 20^{th} aspect according to the preceding aspect 19, if k_{circ} is less than k_{circ}^{ref}, then the reference catheter pressure drop coefficient k_{cath}^{ref} is updated as k_{cath}^{ref}=k_{circ}−kₗᵢₙₑ^{design}.

In a 21^{st} aspect according the preceding aspect 20, if k_{circ} is less than k_{circ}^{ref}, then the reference circuit pressure drop coefficient k_{circ}^{ref} is updated as k_{circ}^{ref}=k_{circ}.

In a 22^{nd} aspect according to any of the preceding aspects 12 to 21, the control unit is configured for or the method comprises: receiving a blood hematocrit Hct and calculating the blood viscosity μ from the blood hematocrit Hct.

In a 23^{rd} aspect according to the preceding aspect 22, the control unit is configured for or the method comprises: receiving a blood temperature T and calculating the blood viscosity μ from the blood hematocrit Hct and the blood temperature T.

In a 23^{rd} bis aspect according to the preceding aspect 22 or 23, the control unit is configured for or the method comprises: receiving a protein or albumin concentration Cp and calculating the blood viscosity μ also from the protein or albumin concentration Cp.

In a 24^{th} aspect according to any one of the preceding aspects 1 to 23, setting the pressure alarm threshold Pₜₕᵣₑₛₕ comprises: setting the pressure alarm threshold Pₜₕᵣₑₛₕ equal to the disconnection pressure P_{disc}.

In a 25^{th} aspect according to any one of the preceding aspects 1 to 23, setting the pressure alarm threshold Pₜₕᵣₑₛₕ Comprises: setting the pressure alarm threshold Pₜₕᵣₑₛₕ equal to the disconnection pressure P_{disc} plus or minus a safety margin ΔP_{safety}.

In a 26^{th} aspect according to the preceding aspect 24 or 25, the control unit is configured for or the method comprises: sending an alarm or a warning signal and/or stopping the extracorporeal blood treatment if the measured pressure P is outside a pressure range delimited by the pressure alarm threshold Pₜₕᵣₑₛₕ.

In a 27^{th} aspect according one or more of the preceding aspects, the at least one connector comprises a connector of the blood return line, the at least one pressure sensor comprises a return pressure sensor configured to detect a pressure at a measurement location in the blood return line and the control unit is configured for or the method comprises: detecting a disconnection between the connector of the blood return line and the vascular access device; optionally the return pressure sensor is coupled to a deaeration chamber in the blood return line.

In a 28^{th} aspect according to the preceding aspect 27, the hydrostatic pressure difference P_{H_patient} is a hydrostatic pressure difference in the blood return line P_{Hret_patient} due to a difference in height Hᵣₑₜ between the vascular access device and the return pressure sensor.

In a 29^{th} aspect according to the preceding aspect 28, the section pressure drop ΔPₗᵢₙₑ is a section pressure drop in the blood return line ΔP_{ret_line} due to a section of the blood return line from the connector of the blood return line to the return pressure sensor.

In a 30^{th} aspect according to the preceding aspect 29, the disconnection pressure P_{disc} is a return disconnection pressure P_{ret_disc} and is a sum of the hydrostatic pressure difference in the blood return line P_{Hret_patient} and the section pressure drop in the blood return line ΔP_{ret_line}.

In a 31^{st} aspect according to the preceding aspect 30, the pressure alarm threshold Pₜₕᵣₑₛₕ is a pressure alarm threshold of the blood return line P_{ret_thresh} and is set as a function of the return disconnection pressure P_{ret_disc}.

In a 32^{nd} aspect according to the preceding aspect 31, the measured pressure P is a measured return pressure Pᵣₑₜ from the return pressure sensor.

In a 33^{rd} aspect according to the preceding aspect 32, the disconnection between the connector of the blood return line and the vascular access device is detected by comparing the measured return pressure Pᵣₑₜ with the pressure alarm threshold of the blood return line P_{ret_thresh}.

In a 34^{th} aspect according to the preceding aspect 33, the control unit is configured for or the method comprises: sending an alarm or a warning signal and/or stopping the extracorporeal blood treatment if the measured return pressure Pᵣₑₜ is equal to or lower than the pressure alarm threshold P_{ret_thresh}.

In a 35^{th} aspect according one or more of the preceding aspects, the at least one connector comprises a connector of the blood withdrawal line, the at least one pressure sensor comprises a withdrawal pressure sensor configured to detect a pressure at a measurement location in the blood withdrawal line and the control unit is configured for or the method comprises: detecting a disconnection between the connector of the blood withdrawal line and the vascular access device.

In a 36^{th} aspect according to the preceding aspect 35, the hydrostatic pressure difference P_{H_patient} is a hydrostatic pressure difference in the blood withdrawal line P_{Hwith_patient} due to a difference in height H_{with} between the vascular access device and the withdrawal pressure sensor.

In a 37^{th} aspect according to the preceding aspect 36, the section pressure drop ΔPₗᵢₙₑ is a section pressure drop in the blood withdrawal line ΔP_{with_line} due to a section of the blood withdrawal line from the connector of the blood withdrawal line to the withdrawal pressure sensor.

In a 38^{th} aspect according to the preceding aspect 37, the disconnection pressure P_{disc} is a withdrawal disconnection pressure P_{with_disc} and is a difference between the hydrostatic pressure difference in the blood withdrawal line P_{Hwith_patient} and the section pressure drop in the blood withdrawal line ΔP_{with_line}.

In a 39^{th} aspect according to the preceding aspect 38, the pressure alarm threshold Pₜₕᵣₑₛₕ is a pressure alarm threshold of the blood withdrawal line P_{with_thresh} and is set as a function of the withdrawal disconnection pressure P_{with_disc}.

In a 40^{th} aspect according to the preceding aspect 39, the measured pressure P is a measured withdrawal pressure P_{with} from the withdrawal pressure sensor.

In a 41^{st} aspect according to the preceding aspect 40, the disconnection between the connector of the blood withdrawal line and the vascular access device is detected by comparing the measured withdrawal pressure P_{with} with the pressure alarm threshold of the blood withdrawal line P_{with_thresh}.

In a 42^{nd} aspect according to the preceding aspect 41, the control unit is configured for or the method comprises: sending an alarm or a warning signal and/or stopping the extracorporeal blood treatment if the measured withdrawal pressure P_{with} is equal to or greater than the pressure alarm threshold P_{with_thresh}.

In a 43^{rd} aspect according to aspects 27 to 34, in order to calculate the section pressure drop in the blood return line Δ_{Pret_line}, the control unit is configured for or the method comprises:
receiving the blood flow rate Qb;
receiving or calculating the blood viscosity μ;
receiving or calculating a section pressure drop coefficient of the blood return line k_{ret_line} function of a geometry of the section of the blood return line;
calculating the pressure drop in the blood return line ΔP_{ret_line} as function of the blood flow rate Qb, the blood viscosity μ and said section pressure drop coefficient of the blood return line k_{ret_line}.

In a 44^{th} aspect according to the preceding aspect 43, the control unit is configured for or the method comprises: updating the section pressure drop coefficient of the blood return line k_{ret_line} and consequently also the return disconnection pressure P_{ret_disc} during the extracorporeal blood treatment.

In a 45^{th} aspect according to the preceding aspect 44, an initial section pressure drop coefficient of the blood return line k_{ret_line}^{init} at the start of the extracorporeal blood treatment is given, optionally by the following equation: k_{ret_line}^{init}=(128×Lᵣₑₜ)/(π×dᵣₑₜ⁴), and it is set as design section pressure drop coefficient of the blood return line k_{ret_line}^{design}; where Lᵣₑₜ is a length of the section of the blood return line and dᵣₑₜ is an internal diameter of the section of the blood return line.

In a 45^{th} bis aspect according to the preceding aspect 45, the initial section pressure drop coefficient of the blood return line k_{ret_line}^{init} at the start of the extracorporeal blood treatment is derived from experimental measurements.

In a 46^{th} aspect according to the preceding aspect 45 or 45 bis, in order to update the section pressure drop coefficient of the blood return line k_{ret_line} during the extracorporeal blood treatment, the control unit is configured for or the method comprises:
calculating an initial circuit pressure drop coefficient of the blood return line k_{ret_circ}^{init} as k_{ret_circ}^{init}=(Pᵣₑₜ^{init}−P_{Qb0})/(μ×Qb) and set it as reference circuit pressure drop coefficient of the blood return line k_{ret_circ}^{ref};
calculating an initial catheter pressure drop coefficient of the blood return line k_{ret_cath}^{init} as k_{ret_cath}^{init}=k_{ret_circ}^{init}−k_{ret_line}^{design} and set it as reference catheter pressure drop coefficient of the blood return line k_{ret_cath}^{ref};
during the extracorporeal blood treatment, measuring the pressure return line Pᵣₑₜ and calculating the circuit pressure drop coefficient of the blood return line as k_{ret_circ}=(Pᵣₑₜ−P_{Qb0})/(μ×Qb);
comparing each new value of the circuit pressure drop coefficient of the blood return line k_{ret_circ} to the reference circuit pressure drop coefficient of the blood return line k_{ret_circ}^{ref}.

In a 46^{th} bis aspect, the circuit pressure drop coefficient k_{ret_circ} of the blood return line is the coefficient related to the pressure drop due to the vascular access device and to the section of the blood return line from the connector of the blood return line to said return pressure sensor.

In a 47^{th} aspect according to the preceding aspect 46, if k_{ret_circ} is greater than k_{ret_circ}^{ref}, then the section pressure drop coefficient of the blood return line k_{ret_line} is updated as k_{ret_line}=k_{ret_circ}−k_{ret_cath}^{ref} and the return disconnection pressure P_{ret_disc} is updated accordingly.

In a 48^{th} aspect according to the preceding aspect 46, if k_{ret_circ} is less than k_{ret_circ}^{ref}, then the section pressure drop coefficient of the blood return line k_{ret_line} remains unchanged and the return disconnection pressure P_{ret_disc} remains unchanged.

In a 49^{th} aspect according to the preceding aspect 48, if k_{ret_circ} is less than k_{ret_circ}^{ref}, then the reference catheter pressure drop coefficient of the blood return line k_{ret_cath}^{ref} is updated as k_{ret_cath}^{ref}=k_{ret_circ}−k_{ret_line}^{design}.

In a 50^{th} aspect according the preceding aspect 49, if k_{ret_circ} is less than k_{ret_circ}^{ref}, then the reference circuit pressure drop coefficient of the blood return line k_{ret_circ}^{ref} is updated as k_{ret_circ}^{ref}=k_{ret_circ}.

In a 51^{st} aspect according to aspects 35 to 42, in order to calculate the section pressure drop in the blood withdrawal line ΔP_{with_line}, the control unit is configured for or the method comprises:
receiving the blood flow rate Qb;
receiving or calculating the blood viscosity μ;
receiving or calculating a section pressure drop coefficient of the blood withdrawal line k_{with_line} function of a geometry of the section of the blood withdrawal line;
calculating the pressure drop in the blood withdrawal line ΔP_{with_line} as function of the blood flow rate Qb, the blood viscosity μ and said section pressure drop coefficient of the blood withdrawal line k_{with_line}.

In a 52^{nd} aspect according to the preceding aspect 51, the control unit is configured for or the method comprises: updating the section pressure drop coefficient of the blood withdrawal line k_{with_line} and consequently also the withdrawal disconnection pressure P_{with_disc} during the extracorporeal blood treatment.

In a 53^{rd} aspect according to the preceding aspect 52, an initial section pressure drop coefficient of the blood withdrawal line k_{with_line}^{init} at the start of the extracorporeal blood treatment is given, optionally by the following equation: k_{with_line}^{init}=(128×L_{with})/(π×d_{with}⁴), and it is set as design section pressure drop coefficient of the blood withdrawal line k_{with_line}^{design}; where L_{with} is a length of the section of the blood withdrawal line and d_{with} is an internal diameter of the section of the blood withdrawal line.

In a 53^{rd} bis aspect according to the preceding aspect 53 or 53 bis, the initial section pressure drop coefficient of the blood withdrawal line k_{with_line}^{init} at the start of the extracorporeal blood treatment is derived from experimental measurements.

In a 54^{th} aspect according to the preceding aspect 53, in order to update the section pressure drop coefficient of the blood withdrawal line k_{with_line} during the extracorporeal blood treatment, the control unit is configured for or the method comprises:
calculating an initial circuit pressure drop coefficient of the blood withdrawal line k_{with_circ}^{init} as k_{with_circ}^{init}=(P_{Qb0}−P_{with}^{init})/(μ×Qb) and set it as reference circuit pressure drop coefficient of the blood withdrawal line k_{with_circ}^{ref};
calculating an initial catheter pressure drop coefficient of the blood withdrawal line k_{with_cath}^{init} as k_{with_cath}^{init}=k_{with_circ}^{init}−k_{with_line}^{design} and set it as reference catheter pressure drop coefficient of the blood withdrawal line k_{with_cath}^{ref};
during the extracorporeal blood treatment, measuring the pressure withdrawal line P_{with} and calculating the circuit pressure drop coefficient of the blood withdrawal line as k_{with_circ}=(P_{with}−P_{Qb0})/(μ×Qb);
comparing each new value of the circuit pressure drop coefficient of the blood withdrawal line k_{with_circ} to the reference circuit pressure drop coefficient of the blood withdrawal line k_{with_circ}^{ref}.

In a 54^{th} bis aspect, the circuit pressure drop coefficient k_{with_circ} of the blood withdrawal line is the coefficient related to the pressure drop due to the vascular access device and to the section of the blood withdrawal line from the connector of the blood withdrawal line to said withdrawal pressure sensor.

In a 55^{th} aspect according to the preceding aspect 54, if k_{with_circ} is greater than k_{with_circ}^{ref}, then the section pressure drop coefficient of the blood withdrawal line k_{with_line} is updated as k_{with_line}=k_{with_circ}−k_{with_cath}^{ref} and the withdrawal disconnection pressure P_{with_disc} is updated accordingly.

In a 56^{th} aspect according to the preceding aspect 54, if k_{with_circ} is less than k_{with_circ}^{ref}, then the section pressure drop coefficient of the blood withdrawal line k_{with_line} remains unchanged and the withdrawal disconnection pressure P_{with_disc} remains unchanged.

In a 57^{th} aspect according to the preceding aspect 56, if k_{with_circ} is less than k_{with_circ}^{ref}, then the reference catheter pressure drop coefficient of the blood withdrawal line k_{with_cath}^{ref} is updated as k_{with_cath}^{ref}=k_{with_circ}−k_{with_line}^{design}.

In a 58^{th} aspect according the preceding aspect 57, if k_{with_circ} is less than k_{with_circ}^{ref}, then the reference circuit pressure drop coefficient of the blood withdrawal line k_{with_circ}^{ref} is updated as k_{with_circ}^{ref}=k_{with_circ}.
In a 59^{th} aspect, the withdrawal disconnection pressure P_{with_disc} calculated through aspects 36, 37 and 38 is the withdrawal disconnection pressure P_{with_disc} immediately after disconnection (e.g. after 1 s to 2 s) while the withdrawal disconnection pressure P_{with_disc} after an amount of time (typically 10 s to 20 s, i.e. the time to empty the line which relates to its (blood) volume V and operating blood flow rate Qb), when the withdrawal line has been filled with air, is set equal to zero (P_{with_disc}=0).

In a 60^{th} aspect according to any of the preceding aspects, the access device comprises a catheter, optionally a double lumen catheter.

In a 61^{st} aspect according the previous aspect, the catheter comprises a withdrawal port connected or configured to be connected to the connector of the blood withdrawal line and a return port connected or configured to be connected to the connector of the blood return line.

In a 62^{nd} aspect according to any of the previous aspects, the apparatus is designed for continuous renal replacement therapies (CRRT) or other therapies in Intensive Care Units (ICU), like Extra-Corporeal CO2 Removal (ECCO2R), Hemo-Perfusion (HP) or Therapeutic Plasma Exchange (TPE).

In a 62^{nd} bis aspect according to any of the previous aspects, the filtration unit has a primary chamber and a secondary chamber separated by a semi-permeable membrane; wherein the blood withdrawal line is connected to an inlet of the primary chamber and the blood return line is connected to an outlet of the primary chamber.

In a 63^{rd} aspect according to any of the previous aspects, the apparatus comprises an effluent line connected to the filtration unit; an effluent pump is configured to be coupled to a pump section of the effluent line.

In a 64^{th} aspect according to any of the previous aspects, the apparatus comprises a dialysis line connected to the filtration unit and to a source of a dialysis fluid; a dialysis pump is configured to be coupled to a pump section of the dialysis line.

In a 65^{th} aspect according to any of the previous aspects, the apparatus comprises at least one infusion line connected to the blood circuit and to a source of at least one infusion fluid; an infusion pump is configured to be coupled to a pump section of said at least one infusion line and to deliver an infusion flow rate.

In a 66^{th} aspect according to any of the previous aspects from 63 to 65 and to aspect 9, the static pressure P_{Qb0} is received or measured after stopping the effluent pump or keeping the effluent pump stopped and/or stopping the dialysis pump or keeping the dialysis pump stopped and/or stopping the infusion pump/s or keeping the infusion pump/s stopped.

### DESCRIPTION OF THE DRAWINGS

Aspects of the invention are shown in the attached drawings, which are provided by way of non-limiting examples, wherein:

FIG. 1 shows a schematic representation of an extracorporeal blood treatment apparatus;

FIG. 2 shows a schematic view in side elevation of the extracorporeal blood treatment apparatus of FIG. 1;

FIG. 3 shows an element of the extracorporeal blood treatment apparatus of FIGS. 1 and 2;

FIG. 4 is a flowchart showing a method of detecting disconnection events in an apparatus for extracorporeal blood treatment according to aspects of the invention;

FIGS. 5 and 6 represent a flowchart showing part of another method of detecting disconnection events in an apparatus for extracorporeal blood treatment according to aspects of the invention;

FIG. 7 is a flowchart showing another part of the method of FIGS. 5 and 6.

### DETAILED DESCRIPTION

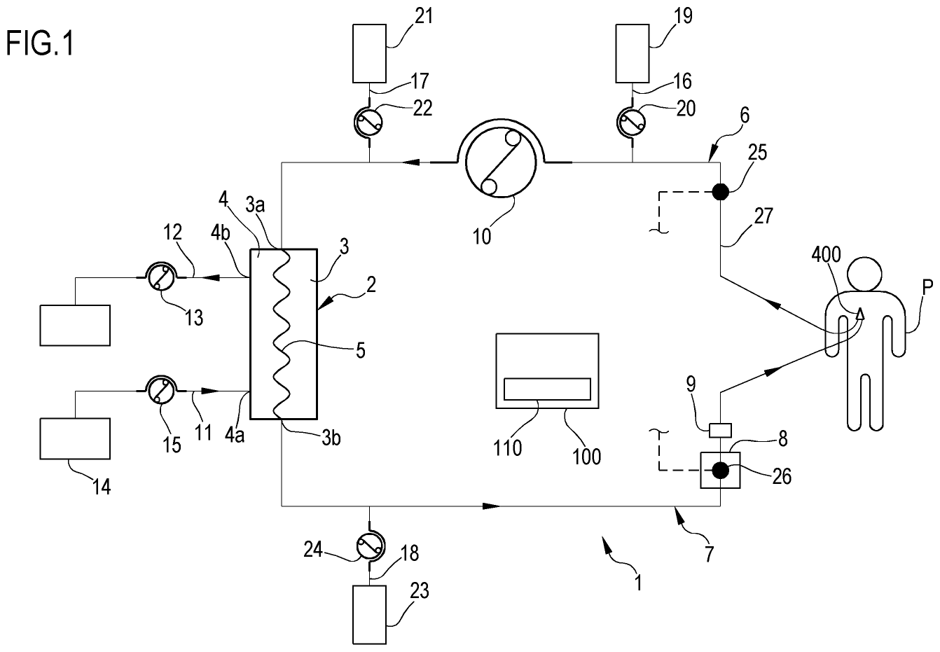

Extracorporeal blood treatment apparatus An apparatus 1 for extracorporeal blood treatment is schematically represented in FIG. 1. The apparatus 1 is a continuous renal replacement therapy (CRRT) apparatus for intensive care treatments, for instance configured to deliver various therapies, like CCVH, CVVHDF, CVVHD, SCUF.

The apparatus 1 comprises a treatment or filtration unit 2 having a primary chamber 3 and a secondary chamber 4 separated by a semi-permeable membrane 5. Depending upon the treatment, the semi-permeable membrane 5 of the filtration unit 2 may be selected to have different properties and performances. A blood circuit is coupled to the primary chamber 3 of the filtration unit 2. The blood circuit comprises a blood withdrawal line 6 connected to an inlet 3a of the primary chamber 3, a blood return line 7 connected to an outlet 3b of the primary chamber 3. The blood withdrawal line 6 and blood return line 7 are configured for connection to a cardiovascular system of a patient “P”.

In use, the blood withdrawal line 6 and the blood return line 7 are connected to a vascular access device 400 which is then placed in fluid communication with the patient “P” vascular system, such that blood may be withdrawn through the blood withdrawal line 6, flown through the primary chamber 3 and then returned to the patient's vascular system through the blood return line 7.

An air detector, not shown, and an air separator, such as a deaeration chamber 8, may be present on the blood return line 7. Moreover, a monitor valve 9 may be present on the blood return line 7, downstream the deaeration chamber 8. The blood flow through the blood circuit is controlled by a blood pump 10, for instance a peristaltic blood pump, acting either on the blood withdrawal line 6 or on the blood return line 7. The embodiment of FIG. 1 shows the blood pump 10 coupled to a pump section of the blood withdrawal line 6. A dialysis circuit is connected to the secondary chamber 4 of the filtration unit 2 and comprises a dialysis line 11 connected to an inlet 4a of the secondary chamber 4 and an effluent line 12 connected to an outlet 4b of the secondary chamber 4 and to a drain, not shown.

An effluent pump 13 is located on the effluent line 12 and is able to recall fluid from the second chamber 4. The dialysis line 11 is connected to a source 14, e.g. a bag or a preparation device, of fresh dialysis fluid and a dialysis pump 15 is located on the dialysis line 11 and is able to pump fluid to the second chamber 4.

The apparatus 1 further comprises an infusion circuit comprising at least one infusion line. The infusion circuit shown in the embodiment of FIG. 1 comprises a pre-blood pump line 16, a pre-infusion line 17 and a post-infusion line 18.

The pre-blood pump line 16 is connected to the blood withdrawal line 6 upstream of the blood pump 10 and to a first source 19 of infusion fluid, e.g. a bag. A pre-blood pump 20 is located on the pre-blood pump line 16 and is able to pump fluid from the first source 19 to the blood circuit.

The pre-infusion line 17 is connected to the blood withdrawal line 6 downstream of the blood pump 10 and upstream of the filtration unit 2 and to a second source 21 of infusion fluid, e.g. a bag. A pre-infusion pump 22 is located on the pre-infusion line 17 and is able to pump fluid from the second source 21 to the blood circuit.

The post-infusion line 18 is connected to the blood return line 7 downstream of the filtration unit 2 and to a third source 23 of infusion fluid, e.g. a bag. A post-infusion pump 24 is located on the post-infusion line 18 and is able to pump fluid from the third source 23 to the blood circuit.

The apparatus 1 may also comprise one or more auxiliary line/s, not shown, connected to the blood circuit and to a source of at least one compensation substance or of an anticoagulant, such as potassium or bicarbonate, and a pump or syringe configured to deliver a flow rate of the compensation substance, such as potassium or bicarbonate, etc.

A withdrawal pressure sensor 25 is configured to detect a pressure at a measurement location in the blood withdrawal line 6. A return pressure sensor 26 is configured to detect a pressure at a measurement location in the blood return line 7. The withdrawal pressure sensor 25 and the return pressure sensor 26 may comprise pressure pods in the blood withdrawal line 6 and blood return line 7. The return pressure sensor 26 may be operatively coupled to the deaeration chamber 8, as shown in FIG. 1.

A control unit 100 is connected and controls the blood pump 10, the dialysis pump 15, the effluent pump 13, the pre-blood pump 20, the pre-infusion pump 22 and the post-infusion pump 24 to regulate a blood flow rate “Q_{b}” in the blood circuit, a dialysis flow rate crossing the dialysis line 11, an effluent flow rate crossing the effluent line 12, an infusion flow rate crossing the pre-blood pump line 16, an infusion flow rate crossing the pre-infusion line 17, an infusion flow rate crossing the post-infusion line 18. Through the control of the dialysis flow rate crossing the dialysis line 11 and/or of the effluent flow rate crossing the effluent line 12, the control unit 100 is also configured to control/regulate a filtration flow rate (through the control of the dialysis pump 15 and the effluent pump 13) in the filtration unit 2 and/or a patient fluid removal rate (also through the control of the pre-blood pump 20, the pre-infusion pump 22 and the post-infusion pump 24).

The control unit 100 is also connected to the withdrawal pressure sensor 25 and to the return pressure sensor 26 to receive signals correlated to pressure values from these sensors 25, 26. The withdrawal pressure sensor 25 and the return pressure sensor 26 provide to the control unit 100 signals correlated to pressure in the extracorporeal blood circuit.

The control unit 100 may be an electronic control unit comprising at least a CPU, a memory and input/output devices. The control unit 100 comprises or is connected to an interface 110 configured to display data and/or allow a user to input data. For instance, the interface comprises a display, e.g. a touch screen, and/or buttons or a keyboard.

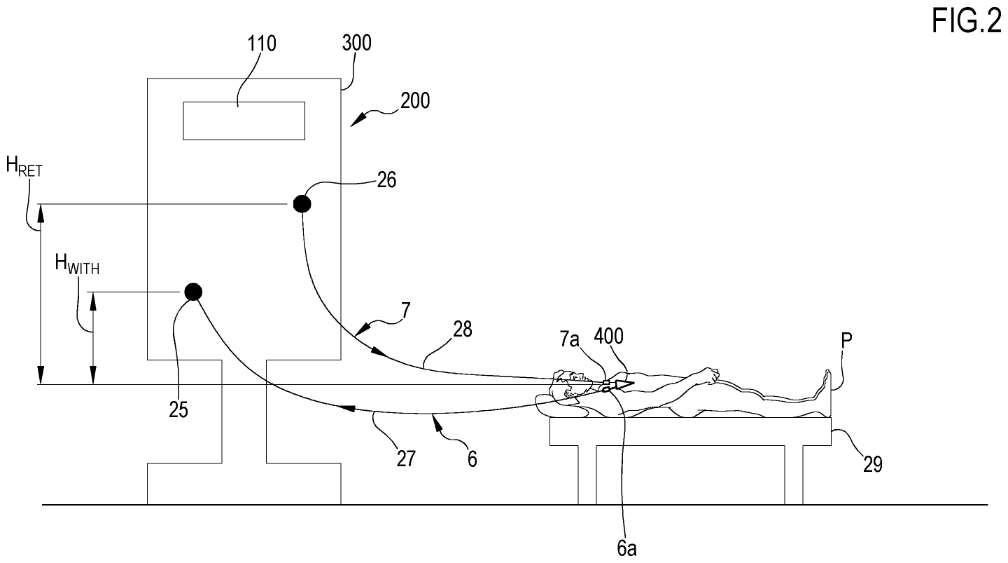

The apparatus 1 may comprise a treatment machine 200 and an integrated disposable set configured to be coupled to the treatment machine 200. The outline of the treatment machine 200 is represented schematically in FIG. 2.

The treatment machine 200 comprises the cited blood pump 10, effluent pump 13, dialysis pump 15, pre-blood pump 20, pre-infusion pump 22, post-infusion pump 24, control unit 100 with the interface 110, flow rate sensors.

The treatment machine 200 may comprise also the withdrawal pressure sensor 25 and the return pressure sensor 26 or the withdrawal pressure sensor 25 and the return pressure sensor 25 may be part of the integrated disposable set. The treatment machine 200 comprises also all the other elements and/or devices configured to receive and hold parts of the integrated disposable set.

The integrated disposable set comprises the treatment or filtration unit 2, the blood circuit, the effluent line 12, the dialysis line 11, the infusion lines 16, 17, 18, which are grouped together.

When the integrated disposable set is mounted on the treatment machine 200, the withdrawal pressure sensor 25 and the return pressure sensor 26 or the measurement locations of the withdrawal pressure sensor 25 and return pressure sensor 26 are in fixed positions on a frame 300 of the treatment machine 200 and at predefined heights above the ground.

A section 27 of the blood withdrawal line 6 develops from the withdrawal pressure sensor 25 on the treatment machine 200 to the vascular access device 400 and to the patient P undergoing treatment. A section 28 of the blood return line 7 develops from the return pressure sensor 26 on the treatment machine 200 to the vascular access device 400 and to the patient P.

As shown in FIG. 2, the patient P undergoing treatment is lying on a bed 29 and the vascular access device 400 is placed at a height which may be different from a height of the withdrawal pressure sensor 25 and of the return pressure sensor 26 and may vary with respect to both the heights of the withdrawal pressure sensor 25 and return pressure sensor 26, since the bed height is typically adjustable.

### Vascular Access Device

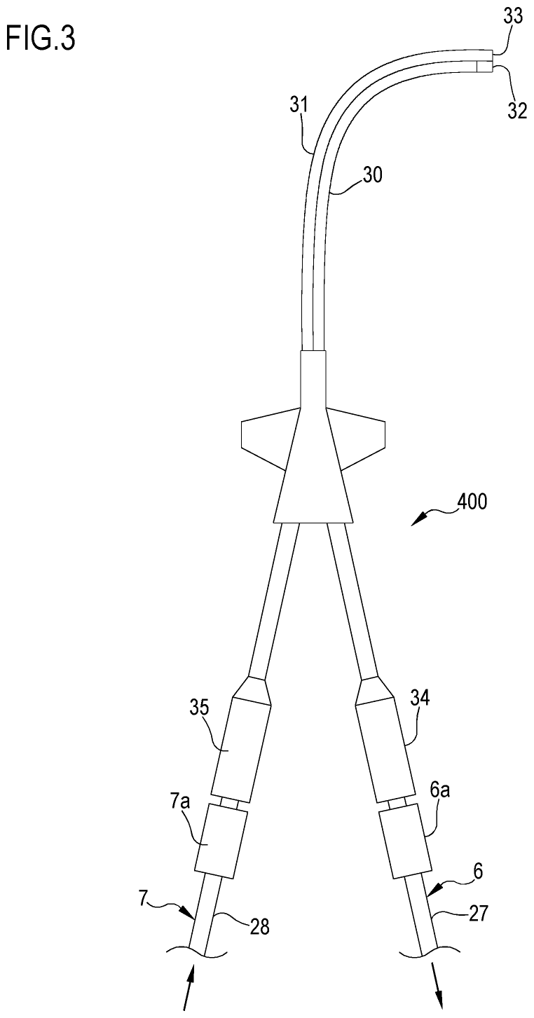

The vascular access device 400 shown in FIG. 3 is central double lumen venous catheter (CVC) which is configured to be placed in a large central vein of the patient P, for instance in the neck (internal jugular vein).

The vascular access device 400 comprises a withdrawal section 30 delimiting a withdrawal lumen and a return section 31 delimiting a return lumen. Distal portions of the withdrawal section 30 and return section 31 are provided respectively with a distal tip 32 of the withdrawal section 30 and a distal tip 33 of the return section. The distal portions of the withdrawal section 30 and return section 31 are paired and configured to be placed inside the large central vein. Proximal portions of the withdrawal section 30 and return section 31 are split and configured to remain outside the patient body.

The proximal portions are provided respectively with a withdrawal port 34 and a return port 35. The withdrawal port 34 is connected or configured to be connected to a connector 6a of the blood withdrawal line 6 and the return port 35 is connected or configured to be connected to a connector 7a of the blood return line 7, as shown in FIG. 3, and allow to connect the extracorporeal blood circuit to the vascular system of the patient P.

### Detection of Disconnection Events

According to a method of detecting disconnection events in an apparatus for extracorporeal blood treatment, the control unit 100 is configured and/or programmed for detecting a disconnection between the withdrawal port 34 and the connector 6a of the blood withdrawal line 6 and/or between the return port 35 and the connector 7a of the blood return line 7 while the patient P is undergoing an extracorporeal blood treatment. When a disconnection event is detected, the control unit 100 is configured and/or programmed for sending an alarm or a warning signal and/or stopping the extracorporeal blood treatment.

### Disconnection of Return Line

During treatment (i.e. when the blood pump 10 is running), the return pressure Pᵣₑₜ at the measurement location of the return pressure sensor 26 is given by the following equation:

$${P}_{ret} = {P}_{offset\_ret} + Δ {P}_{ret\_cath} + Δ {P}_{ret\_line} \qquad Eq . 1 )$$

wherein
P_{offset_ret}=P_{ret_Qb0} is the pressure when there is no blood flow rate (Qb=0);
ΔP_{ret_cath} is the pressure drop in the return section 31 of the vascular access device 400;
ΔP_{ret_line} is the pressure drop in the section 28 of the blood return line 7 from the connector 7a of the blood return line 7 to the return pressure sensor 26.

The return pressure at the measurement location P_{ret_Qb0} when there is no blood flow rate (Qb=0) is given by the following equation:

$${P}_{offset\_ret} = {P}_{ret\_Qb 0} = {P}_{venous} + {P}_{Hret\_patient} \qquad Eq . 2 )$$

wherein
Pᵥₑₙₒᵤₛ is a patient central venous pressure;
P_{Hret_patient} is a hydrostatic pressure difference in the blood return line 7 due to a difference in height Hᵣₑₜ between the vascular access device 400 and the return pressure sensor 26.

Indeed, in the case of zero blood flow rate, there is no pressure drop along the blood circuit and only hydrostatic pressure as well as patient central venous pressure define the offset pressure.

Therefore equation 1 becomes:

$${P}_{ret} = {P}_{venous} + {P}_{Hret\_patient} + Δ {P}_{ret\_cath} + Δ {P}_{ret\_line} \qquad Eq . {1}^{′} )$$

In case of disconnection between the connector 7a of the blood return line 7 and the return port 35 of the vascular access device 400, the return pressure at the measurement location (the return disconnection pressure P_{ret_disc}) is given by the following equation:

$${P}_{ret\_disc} = {P}_{Hret\_patient} + Δ {P}_{ret\_line} \qquad Eq . 3 )$$

because, if the connector 7a is disconnected from the return port 35, the return pressure at the measurement location is not affected by the patient central venous pressure Pᵥₑₙₒᵤₛ and by the pressure drop ΔP_{ret_cath} in the return section of the vascular access device 400.

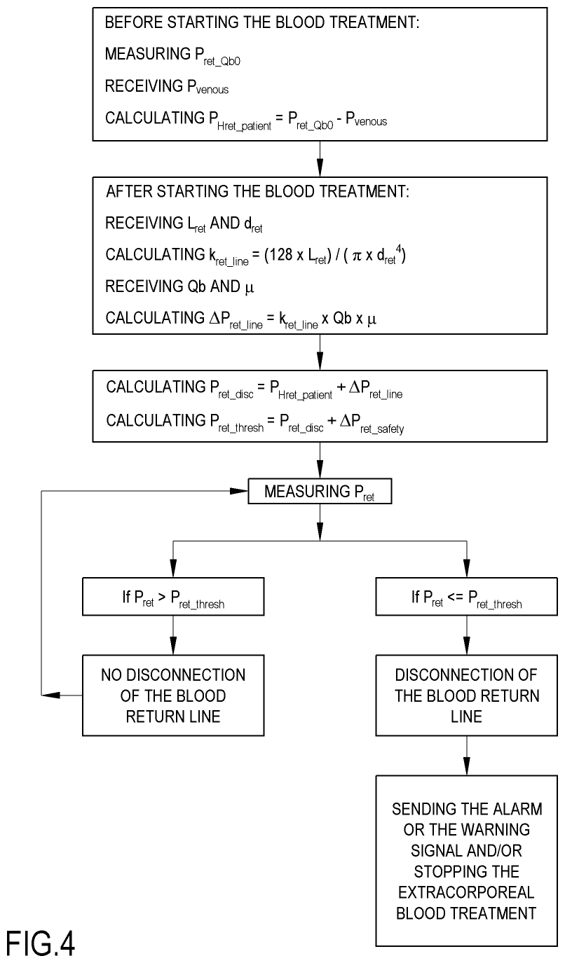

### Example 1 (FIG. 4)

In view of equations 1), 1′), 2) and 3) above, in order to detect the disconnection of the blood return line 7, before starting the blood treatment and after the patient has been connected, while the blood pump 10, the effluent pump 13, the dialysis pump 15, the pre-blood pump 20, the pre-infusion pump 22 and the post-infusion pump 24 are stopped and the blood flow rate is zero (Qb0), the control unit 100 is configured for performing the following steps:
measuring the return pressure P_{ret_Qb0} at the measurement location;
receiving the patient central venous pressure Pᵥₑₙₒᵤₛ; and
calculating the hydrostatic pressure difference P_{Hret_patient} in the blood return line 7 starting from equation 2) as:

$${P}_{Hret\_patient} = {P}_{ret\_Qb 0} - {P}_{v e n o u s} \qquad Eq . 4 )$$

In equation 4), the central venous pressure Pᵥₑₙₒᵤₛ may be set as a default value, for instance between +6 mmHg and +12 mmHg, e.g. of +10 mmHg, because typical values are reported in the 6-12 mmHg range. The central venous pressure Pᵥₑₙₒᵤₛ may also be measured and entered into the control unit 100 through a query to the operator/medical staff.

After starting the blood treatment, the control unit 100 is configured for performing the following steps:
receiving the blood flow rate Qb (e.g. 200 ml/min);
receiving a blood viscosity μ (e.g. 3 mPa s);
receiving a length Lᵣₑₜ (e.g. 2.2 m) and an internal diameter dᵣₑₜ (e.g. 4.5 mm) of the section 28 of the blood return line 7;
calculating a section pressure drop coefficient of the blood return line k_{ret_line} through the following equation:

$${k}_{ret\_line} = ( 128 \times {L}_{ret} ) / ( π \times {d}_{ret}^{4} ) ( e . g . 0.0277 mmHg / ( m 1 / min ) / ( mPa s ) ) \qquad Eq . 5 )$$

calculating the pressure drop ΔP_{ret_line} in the section 28 of the blood return line 7 through the following equation:

$$Δ {P}_{ret\_line} = {k}_{ret\_line} \times Qb \times μ ( e . g . 17 mmHg ) \qquad Eq . 6 )$$

calculating the return disconnection pressure P_{ret_disc} through equation 3 (e.g. 2 mmHg assuming Pᵥₑₙₒᵤₛ=10 and P_{ret_Qb0}=−5 mmHg);
setting a pressure alarm threshold P_{ret_thresh} of the blood return line 7 as:

$${P}_{ret\_thresh} = {P}_{ret\_disc} + Δ {P}_{ret\_safety} ( safety margin ) ( e . g . 12 mmHg assuming a safety margin of 10 mmHg ) \qquad Eq . 7 )$$

Once the pressure alarm threshold P_{ret_thresh} has been calculated, the control unit 100 is configured for performing the following steps:
receiving the measured return pressure Pᵣₑₜ and comparing with the pressure alarm threshold P_{ret_thresh} of the blood return line 7 (with a second or a few seconds frequency);
sending the alarm or the warning signal and/or stopping the extracorporeal blood treatment if the measured return pressure Pᵣₑₜ is equal to or lower than the pressure alarm threshold P_{ret_thresh}.

If Pᵣₑₜ>P_{ret_thresh} no disconnection of the blood return line 7;

If Pᵣₑₜ<=P_{ret_thresh} disconnection of the blood return line 7.

In this Example 1, the section pressure drop coefficient k_{ret_line} of the blood return line 7 is a constant along the extracorporeal blood treatment. If the blood flow rate Qb and the blood viscosity μ remain unchanged, also the pressure drop ΔP_{ret_line} calculated through equation 6) remains unchanged even if the calculation is repeated.

The hydrostatic pressure difference P_{Hret_patient} may change due to changes of bed height and/or of patient position. Therefore, the calculation of the hydrostatic pressure difference P_{Hret_patient}, which is performed before starting the blood treatment through equation 4), is repeated every 6 to 12 hours along the treatment and each time a change in bed height and/or patient position is reported.

In order to repeat the measurements of the return pressure P_{ret_Qb0} and the calculation of the hydrostatic pressure difference P_{Hret_patient}, the pumps (the blood pump 10, the effluent pump 13, the dialysis pump 15, the pre-blood pump 20, the pre-infusion pump 22 and the post-infusion pump 24) are stopped and the static pressure P_{Qb0} is measured when becoming stable.

Then, the return disconnection pressure P_{ret_disc} and the pressure alarm threshold P_{ret_thresh} are updated accordingly. The safety margin ΔP_{ret_safety} may be e.g. 10 mmHg or may be also set to zero, such that the pressure alarm threshold P_{ret_thresh} of the blood return line 7 is equal to the return disconnection pressure P_{ret_disc}.

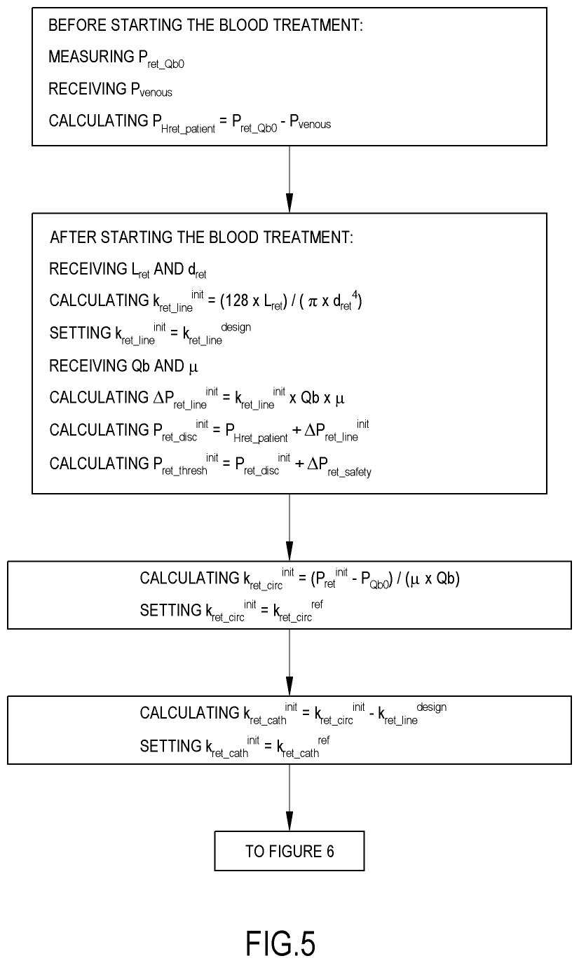

### Example 2 (FIGS. 5, 6 and 7)

According to Example 2, the section pressure drop coefficient k_{ret_line} of the blood return line 7 and consequently also the pressure drop ΔP_{ret_line} in the section 28 of the blood return line 7, the return disconnection pressure P_{ret_disc} and the pressure alarm threshold P_{ret_thresh} are continuously updated during the extracorporeal blood treatment (e.g. every 1 to 10 minutes). This example 2 allows to take into account the presence of clotting in the blood return line 7, since the catheter pressure drop coefficient k_{ret_cath} and the section pressure drop coefficient k_{ret_line} of the blood return line 7 may change along treatment further to clotting problems.

At the start of the extracorporeal blood treatment, the control unit 100 is configured for performing the following steps:
receiving the blood flow rate Qb (e.g. 150 ml/min);
receiving the blood viscosity μ (e.g. 3.0 mPa s);
receiving the length Lᵣₑₜ (e.g. 246 cm) and the internal diameter dᵣₑₜ (e.g. 4.55 mm) of the section 28 of the blood return line 7;
calculating an initial section pressure drop coefficient k_{ret_line}^{init} of the blood return line 7 through the following equation (the same as Eq.5; indeed when initiating blood circulation with a new disposable set, it is safe to assume that no clot is present in the blood return line; in this situation the initial section pressure drop coefficient is provided by Eq. 5):

$${k}_{ret\_line}^{init} = ( 128 \times {L}_{ret} ) / ( π \times {d}_{ret}^{4} ) ( e . g . 0.031 mmHg / ( m 1 / min ) / ( mPa s ) ) \qquad Eq . 8 )$$

calculating ΔP_{ret_line}^{init}=k_{ret_line}^{init}×Qb×μ (e.g. 14 mmHg);
calculating P_{ret_disc}^{init}=P_{Hret_patient}+ΔP_{ret_line}^{init} (e.g. 0 mmHg);
calculating P_{ret_thresh}^{init}=P_{ret_disc}^{init}+ΔP_{ret_safety} (e.g. +10 mmHg);
setting the initial section pressure drop coefficient k_{ret_line}^{init} as design section pressure drop coefficient of the blood return line: k_{ret_line}^{init}=k_{ret_line}^{design};
calculating an initial circuit pressure drop coefficient k_{ret_circ}^{init} of the blood return line 7 through the following equation:

$${k}_{ret\_circ}^{init} = ( {P}_{ret}^{init} - {P}_{Qb 0} ) / ( μ \times Qb ) ( e . g . 0.142 mmHg / ( m 1 / min ) / ( mPa s ) ) \qquad Eq . 9 )$$

setting the initial circuit pressure drop coefficient k_{ret_circ}^{init} as reference circuit pressure drop coefficient k_{ret_circ}^{ref} of the blood return line 7;
calculating an initial catheter pressure drop coefficient k_{ret_cath}^{init} of the blood return line 7 through the following equation:

$${k}_{ret \_ cath}^{init} = {k}_{ret\_circ}^{init} - {k}_{ret\_line}^{design} ( e . g . 0.111 mm Gh / ( ml / min ) / ( mPa s ) ) ) \qquad Eq . 10 )$$

setting the initial catheter pressure drop coefficient k_{ret_cath}^{init} as reference catheter pressure drop coefficient k_{ret_cath}^{ref} of the blood return line 7.

The circuit pressure drop coefficient k_{ret_circ} of the blood return line 7 is the coefficient related to the pressure drop due both to the return section 31 of the vascular access device 400 and to the section 28 of the blood return line 7 from the connector 7a of the blood return line 7 to the return pressure sensor 26. In other embodiments, the initial section pressure drop coefficient of the blood return line k_{ret_line}^{init} at the start of the extracorporeal blood treatment may also be derived from experimental measurements (measurements (in the scenario where the system may identify the return line configuration, either unique or in relation to the set type).

During the extracorporeal blood treatment, the control unit 100 is configured for performing the following steps:
measuring the pressure return line Pᵣₑₜ;
calculating the circuit pressure drop coefficient k_{ret_circ} of the blood return line 7 through the following equation:

$${k}_{ret \_ circ} = ( {P}_{ret} - {P}_{Qb 0} ) / ( μ \times Qb ) \qquad Eq . 11 )$$

comparing each new value of the circuit pressure drop coefficient k_{ret_circ} of the blood return line 7 to the reference circuit pressure drop coefficient k_{ret_circ}^{ref} of the blood return line 7; and then
   if k_{ret_circ} is greater than k_{ret_circ}^{ref}, then the section pressure drop coefficient k_{ret_line} of the blood return line 7 is updated as

$${k}_{ret \_ line} = {k}_{ret \_ circ} - {k}_{ret \_ cath}^{ref} \qquad Eq . 12 )$$

and the return disconnection pressure P_{ret_disc} is updated accordingly through equations 6, 3 and 7;
if k_{ret_circ} is less than or equal to k_{ret_circ}^{ref}, then the section pressure drop coefficient k_{ret_line} of the blood return line 7 remains unchanged and also the return disconnection pressure P_{ret_disc} remains unchanged while the reference catheter pressure drop coefficient k_{ret_cath}^{ref} of the blood return line 7 is updated design and the reference circuit as k_{ret_cath}^{ref}=k_{ret_circ}−k_{ret_line} pressure drop coefficient k_{ret_circ}^{ref} of the blood return line 7 is updated as k_{ret_circ}^{ref}=k_{ret_circ}.

In the situation where k_{ret_circ} is less than k_{ret_circ}^{ref}, the circuit pressure drop coefficient k_{ret_circ} of the blood return line 7 is lower than ever documented since the start of the treatment. This means that the catheter pressure drop was initially overestimated, expectedly because of some clotting that has resolved over time, assuming that the section pressure drop coefficient k_{ret_line} of the blood return line 7 cannot decrease below its initial value.

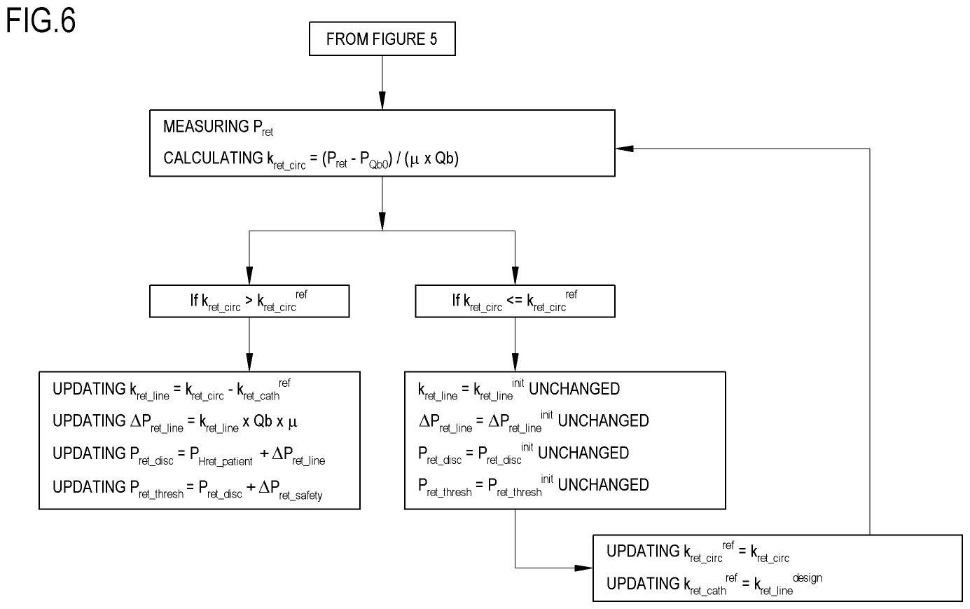

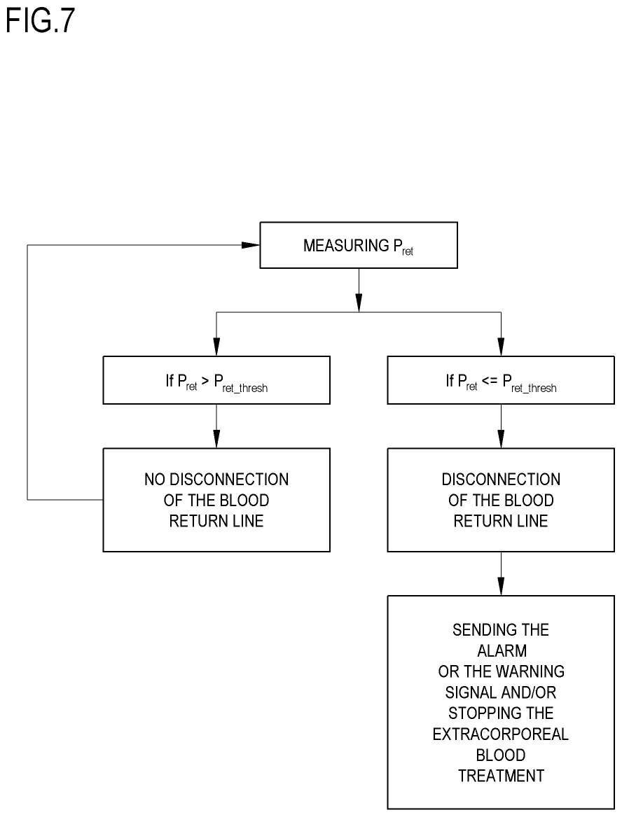

The procedure of FIGS. 5 and 6 is repeated with a period in the range of minutes, e.g. 1 to 10 minutes, while the control procedure of FIG. 7, corresponding to the last part of the method of Example 1 shown in FIG. 4, is repeated with a period in the range of seconds or less, e.g. 0.2 to 2 seconds.

The following Table 1 is an example of evolution of the pressure return line Pᵣₑₜ over time and related computed pressure drop coefficients and alarm threshold obtained starting from the above illustrative values between brackets of this example 2.

TABLE 1

| Time | Qb | Pᵣₑₜ | kᵣₑₜ_{_{—}}_{circ} | kᵣₑₜ_{_{—}}ₗᵢₙₑ | kᵣₑₜ_{_{—}}_{circ}^{ref} | kᵣₑₜ_{_{—}}_{cath}^{ref} | Pᵣₑₜ_{_{—}}ₜₕᵣₑₛₕ |
|---|---|---|---|---|---|---|---|
| min | ml/min | mmHg | mmHg/(ml/min)/(mPa s) | | | | mmHg |
| 0 | 150 | 58 | 0.142 | 0.031 | 0.142 | 0.111 | +10 |
| 10 | 150 | 60 | 0.147 | 0.036 | 0.142 | 0.111 | +12 |
| 20 | 150 | 59 | 0.144 | 0.033 | 0.142 | 0.111 | +11 |
| 26⁽¹⁾ | 200 | 81 | 0.145 | 0.034 | 0.142 | 0.111 | +30 |
| 30 | 200 | 83 | 0.148 | 0.037 | 0.142 | 0.111 | +32 |
| 40⁽²⁾ | 200 | 96 | 0.170 | 0.059 | 0.142 | 0.111 | +45 |
| 50⁽²⁾ | 200 | 101 | 0.178 | 0.067 | 0.142 | 0.111 | +50 |
| 60⁽²⁾ | 200 | 103 | 0.182 | 0.070 | 0.142 | 0.111 | +52 |
| 70⁽³⁾ | 200 | 88 | 0.157 | 0.045 | 0.142 | 0.111 | +37 |
| 80⁽³⁾ | 200 | 82 | 0.147 | 0.039 | 0.142 | 0.111 | +34 |
| 90⁽³⁾ | 200 | 77 | 0.138 | 0.031 | 0.138 | 0.107 | +29 |
| 100 | 200 | 81 | 0.145 | 0.038 | 0.138 | 0.107 | +33 |

⁽¹⁾blood flow rate change at time 26 min (values a few seconds after the flow change)
⁽²⁾simulation of some clotting in the return circuit
⁽³⁾recovery from clotting

Once the pressure alarm threshold P_{ret_thresh} has been calculated, the control unit 100 is configured for performing the following steps:
receiving the measured return pressure Pᵣₑₜ and comparing with the pressure alarm threshold P_{ret_thresh} of the blood return line 7 (with a second or a few seconds frequency);
sending the alarm or the warning signal and/or stopping the extracorporeal blood treatment if the measured return pressure Pᵣₑₜ is equal to or lower than the pressure alarm threshold P_{ret_thresh}.

### Example 3

Example 3 may be a variant embodiment of Example 1 or of Example 2 wherein the blood viscosity μ is calculated by the control unit 100.

The control unit 100 is configured for receiving a blood hematocrit Hct and a blood temperature T and for calculating the blood viscosity μ through the following equation:

$$μ = {e}^{( 1 , 8 / T ) \times 5 , 54} \times {e}^{2 , 3 \times ( Hct / 100 )} \qquad Eq . 13 )$$

The hematocrit Hct and the blood temperature T may be entered by an operator in the control unit through the interface 110 or may be measured by sensors operatively connected to the control unit 100 and automatically transmitted to the control unit 100. For instance, an optical sensor may be used to measure hematocrit “Htc”.

### Disconnection of Withdrawal Line

The following equations pertaining to the blood withdrawal line 6 are similar to those related to the return line 7.

$${P}_{with} = {P}_{offset \_ with} - Δ {P}_{with \_ cath} - Δ {P}_{with \_ line} ( see Eq . 1 ) \qquad Eq . 14 )$$

$${P}_{offset \_ with} = {P}_{with \_ Qb 0} = {P}_{venous} + {P}_{Hwith \_ patient} ( see Eq . 2 ) \qquad Eq . 15 )$$

$$Eq . 16 )$$

$${P}_{with} = {P}_{venous} + {P}_{Hwith \_ patient} - Δ {P}_{with \_ cath} - Δ {P}_{with \_ line} ( see Eq . {1}^{′} )$$

$${P}_{with \_ disc} = {P}_{Hwith \_ patient} - Δ {P}_{with \_ line} ( see Eq . 3 ) \qquad Eq . 17 )$$

wherein
P_{with}: measured withdrawal pressure from the withdrawal pressure sensor 25;
P_{with_Qb0}: withdrawal pressure at the measurement location when there is no blood flow rate (Qb=0);
P_{Hwith_patient}: hydrostatic pressure difference in the blood withdrawal line 6 due to a difference in height between the vascular access device 400 and the withdrawal pressure sensor 25;
ΔP_{with_cath}: pressure drop in the withdrawal section 30 of the vascular access device 400;
ΔP_{with_line}: section pressure drop in the blood withdrawal line 6 due to the section 27 of the blood withdrawal line 6 from the connector 6a of the blood withdrawal line 6 to the withdrawal pressure sensor 25;
P_{with_disc}: withdrawal disconnection pressure.

Equation 17 is used to calculate the withdrawal disconnection pressure immediately after a disconnection event.

Differently, since after disconnection of the withdrawal line 6 the blood pump 10 sucks air and the withdrawal line 6 is emptied of blood and filled with air, after an amount of time (e.g. 1 s to 2 s) the withdrawal disconnection pressure P_{with_disc} may be considered negligible and set to zero (P_{with_disc}=0).

### Example 4

Example 4 is analogous to Example 1. In order to detect the disconnection of the blood withdrawal line 6, before starting the blood treatment, while the blood pump 10, the effluent pump 13, the dialysis pump 15, the pre-blood pump 20, the pre-infusion pump 22 and the post-infusion pump 24 are stopped and the blood flow rate is zero (Qb0), the control unit 100 is configured for performing the following steps:
measuring the withdrawal pressure P_{with_Qb0} at the measurement location;
receiving the patient central venous pressure Pᵥₑₙₒᵤₛ; and
calculating the hydrostatic pressure difference PH_{with} patient in the blood withdrawal line 6 starting from equation 15) as:

$${P}_{Hwith \_ patient} = {P}_{with \_ Qb 0} - {P}_{venous} \qquad Eq . 18 )$$

After starting the blood treatment, the control unit 100 is configured for performing the following steps:
receiving the blood flow rate Qb;
receiving the blood viscosity μ (which may be calculated as disclosed in Example 3);
receiving a length L_{with} and an internal diameter d_{with} of the section 27 of the blood withdrawal line 6;
calculating a section pressure drop coefficient of the blood withdrawal line k_{with_line} through the following equation:

$${k}_{with \_ line} = ( 1 \; 28 \times {L}_{with} ) / ( Π \times {d}_{with}^{4} ) ( analogous to Eq . 5 ) \qquad Eq . 19 )$$

calculating the pressure drop ΔP_{with_line} in the section 27 of the blood withdrawal line 6 through the following equation:

$$Δ {P}_{with \_ line} = {k}_{with \_ line} \times Qb \times μ ( analogous to Eq . 6 ) \qquad Eq . 20 )$$

calculating the withdrawal disconnection pressure P_{with_disc} immediately after disconnection through equation 17;
setting a pressure alarm threshold P_{with_thresh} of the blood withdrawal line 6 as:

$${P}_{with \_ thresh} = {P}_{with \_ disc} - Δ {P}_{with \_ safety} ( safety margin ) \qquad Eq . 21 )$$

receiving the measured return pressure P_{with} and comparing with the pressure alarm threshold P_{with_thresh} of the blood withdrawal line 6;
sending the alarm or the warning signal and/or stopping the extracorporeal blood treatment if the measured withdrawal pressure P_{with} is equal to or greater than the pressure alarm threshold P_{with_thresh}.

If P_{with}<P_{with_thresh} no disconnection of the blood withdrawal line 6;

If P_{with}>=P_{with_thresh} disconnection of the blood withdrawal line 6.

### Example 5

Example 5 is analogous to Example 2. According to Example 5, the section pressure drop coefficient k_{with_line} of the blood withdrawal line 6 and consequently also the pressure drop ΔP_{with_line} in the section 27 of the blood withdrawal line 6, the withdrawal disconnection pressure P_{with_disc} and the pressure alarm threshold P_{with_thresh} are continuously updated during the extracorporeal blood treatment.

At the start of the extracorporeal blood treatment, an initial section pressure drop coefficient k_{with_line}^{init} of the blood withdrawal line 6 is calculated through the following equation (same as Eq.19):

$${k}_{with \_ line}^{init} = ( 1 \; 28 \times {L}_{with} ) / ( Π \times {d}_{with}^{4} ) \qquad Eq . 22 )$$

The initial section pressure drop coefficient k_{with_line}^{init} is set as design section pressure drop coefficient k_{with_line}^{design} of the blood withdrawal line 6.

An initial circuit pressure drop coefficient k_{with_circ}^{init} of the blood withdrawal line 7 is calculated through the following equation:

$${k}_{with \_ circ}^{init} = ( {P}_{Qb 0} - {P}_{with}^{init} ) / ( μ \times Qb ) ( analogous to Eq . 9 ) \qquad Eq . 23 )$$

The initial circuit pressure drop coefficient k_{with_circ}^{init} is set as reference circuit pressure drop coefficient k_{with_circ}^{ref} of the blood withdrawal line 6.

An initial catheter pressure drop coefficient k_{with_cath}^{init} of the blood withdrawal line 6 through the following equation:

$${k}_{with \_ cath}^{init} = {k}_{with \_ circ}^{init} - {k}_{with \_ line}^{design} ( analogous to Eq . 10 ) \qquad Eq . 24 )$$

The initial catheter pressure drop coefficient k_{with_cath}^{init} is set as reference catheter pressure drop coefficient k_{with_cath}^{ref} of the blood withdrawal line 6.

During the extracorporeal blood treatment, the control unit 100 measures the pressure withdrawal line P_{with} and calculates the circuit pressure drop coefficient k_{with_circ} of the blood withdrawal line 6 through the following equation:

$${k}_{with \_ circ} = ( {P}_{Qb 0} - {P}_{with} ) / ( μ \times Qb ) ( analogous to Eq . 11 ) \qquad Eq . 25 )$$

Each new value of the circuit pressure drop coefficient k_{with_circ} of the blood withdrawal line 6 is compared to the reference circuit pressure drop coefficient k_{with_circ}^{ref} of the blood withdrawal line 6; and then:

if k_{with_circ} is greater than k_{with_circ}^{ref}, then the section pressure drop coefficient k_{with_line} of the blood withdrawal line 6 is updated as

$${k}_{with \_ line} = {k}_{with \_ circ} - {k}_{with \_ cath}^{ref} ( analogous to Eq . 12 ) \qquad Eq . 26 )$$

and the withdrawal disconnection pressure P_{with_disc} is updated accordingly through equations 20, 17 and 21;
if k_{with_circ} is less than k_{with_circ}^{ref}, then the section pressure drop coefficient k_{with_line} of the blood withdrawal line 6 remains unchanged and also the withdrawal disconnection pressure P_{with_disc} remains unchanged while the reference catheter pressure drop coefficient k_{with_cath}^{ref} of the blood withdrawal line 6 is updated as k_{with_cath}^{ref}=k_{with_circ}−k_{with_line}^{design} and the reference circuit pressure drop coefficient k_{with_circ}^{ref} of the blood withdrawal line 6 is updated as k_{with_circ}^{ref}=k_{with_circ}.

### Example 6

According to Examples 2 and 5, a consistency check of the circuit pressure drop coefficient k_{ret_circ} of the blood return line 7 or of the circuit pressure drop coefficient k_{with_circ} of the blood withdrawal line 7 may also be performed. Equations and computation steps at start and during therapy are unchanged. For instance, referring to Example 2, where the access device 400 (catheter) type is identified and its pressure drop coefficient known (k_{ret_cath}^{design}), the previous algorithm of Example 2 may be slightly tuned. The change consists in an additional check for consistency of the return circuit (or catheter) pressure drop coefficient through the following equation.

$${k}_{ret \_ circ} > {k}_{ret \_ cath}^{design} + {k}_{ret \_ line}^{design} \qquad Eq . 27 )$$

In case above equation is not verified-within the accuracy limits of the measurements-this may drive a confirmation of the catheter type as well as suspicion of return pressure malfunction if the same outcome occurs after repetition of the measuring sequence.

While the invention has been described in connection with what is presently considered to be the most practical and preferred embodiment, it is to be understood that the invention is not to be limited to the disclosed embodiment, but on the contrary, is intended to cover modifications included within the scope of the appended claims.

1-23. (canceled)

24. Apparatus for extracorporeal blood treatment comprising:
a filtration unit;
an extracorporeal blood circuit having a blood withdrawal line connected to an inlet of the filtration unit and a blood return line connected to an outlet of the filtration unit, said extracorporeal blood circuit configured to connect to a cardiovascular system of a patient; the extracorporeal blood circuit comprising at least one connector configured to connect to a vascular access device fixed to the patient;
at least one pressure sensor configured to detect a pressure in at least one measurement location in the extracorporeal blood circuit;
a blood pump configured to control flow of blood through the extracorporeal blood circuit;
a control unit connected to the blood pump and to the at least one pressure sensor, the control unit configured to detect a disconnection between the at least one connector of the extracorporeal blood circuit and the vascular access device by:
calculating a hydrostatic pressure difference due to a difference in height between the vascular access device and said at least one pressure sensor;
calculating a section pressure drop due to a section of the blood circuit from the at least one connector to said at least one pressure sensor;
calculating a disconnection pressure as an algebraic sum of the hydrostatic pressure difference and the section pressure drop;
setting a pressure alarm threshold as a function of the disconnection pressure;
wherein, during an extracorporeal blood treatment, the control unit is configured to:
receive a measured pressure from the at least one pressure sensor;
detect a disconnection of the least one connector from the vascular access device by comparing the measured pressure with the pressure alarm threshold.

25. The apparatus of claim 24, wherein the disconnection pressure is a constant along at least part of the extracorporeal blood treatment or wherein the control unit is configured to continuously update the disconnection pressure during at least part of the extracorporeal blood treatment.

26. The apparatus of claim 24, wherein the control unit is configured to calculate the hydrostatic pressure difference before starting the extracorporeal blood treatment.

27. The apparatus of claim 26, wherein the control unit is configured to calculate the hydrostatic pressure difference at least one time during the extracorporeal blood treatment.

28. The apparatus of claim 24, wherein the control unit is configured to calculate the section pressure drop before starting the extracorporeal blood treatment.

29. The apparatus of claim 28, wherein the control unit is configured to calculate the section pressure drop at least one time the extracorporeal blood treatment.

30. The apparatus of claim 28, wherein the control unit is configured to continuously update the section pressure drop during at least part of the extracorporeal blood treatment.

31. The apparatus of claim 24, wherein, to calculate the hydrostatic pressure difference, the control unit is configured to:
stop the blood pump or keep the blood pump stopped to obtain a zero a blood flow rate;
receive a static pressure from the at least one pressure sensor while the blood flow rate is zero;
receive a patient central venous pressure;
calculate the hydrostatic pressure difference as a difference between the static return pressure and the central venous pressure.

32. The apparatus of claim 31, wherein the patient central venous pressure is measured.

33. The apparatus of claim 31, wherein the patient central venous pressure is set as a default value.

34. The apparatus of claim 33, wherein the default value is between +800 Pa and +1600 Pa or is +1333 Pa.

35. The apparatus of claim 24, wherein, to calculate the section pressure drop, the control unit is configured to:
receive a blood flow rate;
receive or calculate a blood viscosity;
receive or calculate a section pressure drop coefficient function of a geometry of the section of the blood circuit;
calculate the section pressure drop as function of the blood flow rate, the blood viscosity, and said section pressure drop coefficient.

36. The apparatus of claim 35, wherein the section pressure drop coefficient is a constant.

37. The apparatus of claim 35, wherein the section pressure drop coefficient is given by the following equation: kₗᵢₙₑ=(128×L)/(π×d⁴); where L is a length of the section and d is an internal diameter of the section.

38. The apparatus of claim 35, wherein the control unit is configured to update the section pressure drop coefficient and the disconnection pressure during the extracorporeal blood treatment.

39. The apparatus of claim 31, wherein, to calculate the section pressure drop, the control unit is configured to:
receive a blood flow rate;
receive or calculate a blood viscosity;
receive or calculate a section pressure drop coefficient function of a geometry of the section of the blood circuit;
calculate the section pressure drop as function of the blood flow rate, the blood viscosity, and said section pressure drop coefficient, wherein an initial section pressure drop coefficient at a start of the extracorporeal blood treatment is given and is set as design section pressure drop coefficient, where L is a length of the section and d is an internal diameter of the section;
update the section pressure drop coefficient and the disconnection pressure during the extracorporeal blood treatment, wherein, to update the section pressure drop coefficient during the extracorporeal blood treatment, the control unit is configured to:
calculate an initial circuit pressure drop coefficient as k_{circ}^{init}=|(Pᵢₙᵢₜ−P_{Qb0})|/(μ×Qb) and set it as reference circuit pressure drop coefficient;
calculate an initial catheter pressure drop coefficient as k_{cath}^{init}=k_{circ}^{init}−kₗᵢₙₑ^{design} and set it as reference catheter pressure drop coefficient;
during the extracorporeal blood treatment, measure the pressure and calculate the circuit pressure drop coefficient as k_{circ}=(P−P_{Qb0})/(μ×Qb);
compare each calculated value of the circuit pressure drop coefficient to the reference circuit pressure drop coefficient and:
if k_{circ} is greater than k_{circ}^{ref} then the section pressure drop coefficient is updated as kₗᵢₙₑ=k_{circ}−k_{cath}^{ref} and the disconnection pressure is updated accordingly;
if k_{circ} is less than k_{circ}^{ref} then the section pressure drop coefficient remains unchanged and the disconnection pressure remains unchanged; the reference catheter pressure drop coefficient is updated as k_{cath}^{ref}=k_{circ}−kₗᵢₙₑ design, the reference circuit pressure drop coefficient is updated as k_{circ}^{ref}=k_{circ}.

40. The apparatus of claim 39, wherein the initial section pressure drop coefficient at the start of the extracorporeal blood treatment is given by the following equation: kₗᵢₙₑ^{init}=(128×L)/(π×d⁴).

41. The apparatus of claim 35, wherein the control unit is configured to:
receive a blood hematocrit;
calculate the blood viscosity from the blood hematocrit.

42. The apparatus of claim 41, wherein the control unit is configured to:
receive a blood temperature and/or receive one of a protein concentration or albumin concentration;
calculate the blood viscosity also from the blood temperature and/or one of the protein concentration or albumin concentration.

43. The apparatus of claim 24, wherein control unit is configured to set the pressure alarm threshold by:
setting the pressure alarm threshold equal to the disconnection pressure; or
setting the pressure alarm threshold equal to the disconnection pressure plus a safety margin;
wherein the control unit is configured to send an alarm or a warning signal and/or stop the extracorporeal blood treatment when the pressure detected by the at least one pressure sensor in the at least one measurement location is outside a pressure range delimited by the pressure alarm threshold.

44. The apparatus of claim 24, wherein the at least one connector comprises a connector of the blood return line, wherein the at least one pressure sensor comprises a return pressure sensor configured to detect pressure at a measurement location in the blood return line, and wherein the control unit is configured to detect a disconnection between the connector of the blood return line and the vascular access device;
wherein the hydrostatic pressure difference is a hydrostatic pressure difference in the blood return line due to a difference in height between the vascular access device and the return pressure sensor;
wherein the section pressure drop is a section pressure drop in the blood return line due to a section of the blood return line from the connector of the blood return line to the return pressure sensor;
wherein the disconnection pressure is a return disconnection pressure and is a sum of the hydrostatic pressure difference in the blood return line and the section pressure drop in the blood return line;
wherein the pressure alarm threshold is a pressure alarm threshold of the blood return line and is set as a function of the return disconnection pressure;
wherein the pressure detected at the measurement location in the blood return line is a measured return pressure from the return pressure sensor;
wherein the control unit is configured to detect the disconnection between the connector of the blood return line and the vascular access device by comparing the measured return pressure with the pressure alarm threshold of the blood return line.

45. The apparatus of claim 44, wherein control unit is configured to set the pressure alarm threshold by:
setting the pressure alarm threshold equal to the disconnection pressure; or
setting the pressure alarm threshold equal to the disconnection pressure plus a safety margin;
wherein the control unit is configured to send an alarm or a warning signal and/or stop the extracorporeal blood treatment when the measured return pressure is outside a pressure range delimited by the pressure alarm threshold or equal to or lower than the pressure alarm threshold.

46. The apparatus of claim 24, wherein the at least one connector comprises a connector of the blood withdrawal line, wherein the at least one pressure sensor comprises a withdrawal pressure sensor configured to detect pressure at a measurement location in the blood withdrawal line, and wherein the control unit is configured to detect a disconnection between the connector of the blood withdrawal line and the vascular access device;
wherein the hydrostatic pressure difference is a hydrostatic pressure difference in the blood withdrawal line due to a difference in height between the vascular access device and the withdrawal pressure sensor;
wherein the section pressure drop is a section pressure drop in the blood withdrawal line due to a section of the blood withdrawal line from the connector of the blood withdrawal line to the withdrawal pressure sensor;
wherein the disconnection pressure is a withdrawal disconnection pressure and is a difference between the hydrostatic pressure difference in the blood withdrawal line and the section pressure drop in the blood withdrawal line;
wherein the pressure alarm threshold is a pressure alarm threshold of the blood withdrawal line and is set as a function of the withdrawal disconnection pressure;
wherein the pressure detected at the measurement location in the blood withdrawal line is a measured withdrawal pressure from the withdrawal pressure sensor;
wherein the control unit is configured to detect the disconnection between the connector of the blood withdrawal line and the vascular access device by comparing the measured withdrawal pressure with the pressure alarm threshold of the blood withdrawal line.

47. The apparatus of claim 24, designed for continuous renal replacement therapies, Extra-Corporeal CO₂ Removal, Hemo-Perfusion or Therapeutic Plasma Exchange.
